(19) United States
(12) Patent Application Publication
(10) Pub. No.: US 2011/0280862 A1
(43) Pub. Date: Nov. 17, 2011

(54) GEP AND DRUG TRANSPORTER REGULATION, CANCER THERAPY AND PROGNOSIS

(75) Inventors: Siu Tim Cheung, Hong Kong (HK); Sheung Tat Fan, Hong Kong (HK)

(73) Assignee: THE UNIVERSITY OF HONG KONG, Hong Kong (HK)

(21) Appl. No.: 13/107,034

(22) Filed: May 13, 2011

Related U.S. Application Data
(60) Provisional application No. 61/334,671, filed on May 14, 2010.

(51) Int. Cl. A61K 39/395; A61P 43/00; C12N 5/02

(52) U.S. Cl. 424/130.1; 435/375

(57) ABSTRACT

Described herein are methods for manipulating GEP and/or drug transporters (e.g., ABCB5 and/or ABCF1) on a cell, as well as related products. Also described herein are methods for treating cancer cells using GEP and/or drug transporter and/or their binding molecules and suppression thereof. Methods of cancer treatment targeting the GEP and/or drug transporters, alone or in combination with chemotherapy are also described herein. Also provided herein are sets of markers whose expression patterns can be used to differentiate clinical conditions, such as high or low levels of GEP and drug transporters. Based on the levels of GEP and drug transporters, the likelihood of cancer recurrences, drug sensitivity, and prognosis can be determined. Methods of classifying and treating patients based on the prognosis are also provided herein.

### CROSS REFERENCE TO RELATED APPLICATIONS

This application claims priority to U.S. Provisional Patent Application Ser. No. 61/334,671, filed on May 14, 2010, and entitled “GEP AND DRUG TRANSPORTER REGULATION, CANCER THERAPY AND PROGNOSIS,” the entirety of which is incorporated by reference herein.

### TECHNICAL FIELD

This disclosure generally relates to methods for treating hepatic cancers exhibiting chemoresistance by targeting a growth factor (granulin-epithelin precursor (GEP) and an ATP-dependent binding cassette (ABC) drug efflux transporter (ABCB5 or ABCF1) in connection with chemotherapy.

### BACKGROUND

Liver cancer is the third leading cancer killer in the world, with more than half a million individuals dying globally each year. In China, liver cancer is the second major cause of cancer death. Surgical resection, in the form of a partial hepatectomy or a liver transplant, is the mainstay of curative treatment. Nonetheless, cancer recurrence is still common after curative surgery. In addition, liver cancer is frequently diagnosed at an advanced stage, which precludes curative treatment. No effective therapeutic option exists for the treatment of the majority of liver cancer patients. Chemotherapy is widely used to treat unresectable liver cancer, but with marginal efficiency. There is an urgent need to elucidate the key genes in relation to recurrence and chemoresistance in the clinical situation, and to develop a novel therapeutic approach to sensitize liver cancer cells to chemotherapeutic agents.

Multidrug resistance can result from distinct mechanisms, e.g., alterations of tumor cell cycle checkpoints impairment of tumor apoptotic pathways, and reduced drug accumulation in tumor cells. Among these, decreased intracellular drug accumulation is a well-studied mechanism of cancer multidrug resistance and has been shown to result in part from tumor cell expression of the ATP-dependent binding cassette (ABC) drug efflux transporter ABCB1 (also named P-glycoprotein, or MDR1). In the human ABC superfamily, ABCB1 and ABCC1 (also named MRP1) have been shown to mediate multidrug resistance, each with distinct yet overlapping efflux substrate specificities and tissue distribution patterns. The multidrug resistance phenotype was reported in liver carcinogenesis long ago. The phenotype is commonly mediated through overexpression of ABC drug transporters, including ABCB1 and ABCC1. These genes enable liver cancer cells to efflux a broad range of chemically diverse chemotherapeutic agents. Nonetheless, the key genes that regulate chemoresistance in clinical situations have yet to be identified for liver cancer patients.

The contribution of tumorigenic stem cells to hematopoietic cancers has been established for some time, and cells possessing stem cell properties have been described in several solid tumors. Although chemotherapeutic agents would kill most of the tumor cells, they are believed to leave a small population of tumor stem cells behind, which might be an important mechanism of drug resistance. For example, the ABC drug transporters have been shown to protect cancer stem cells from chemotherapy, e.g., ABCB1 in glioblastoma and ABCB5 in melanoma.

Granulin-epithelin precursor (GEP, also named progranulin, proepithelin, acrogranin, or PC-derived growth factor) is a multi-facet autocrine growth factor with different biological roles, including cancer progression, murine fetal development, and tissue repair. Mutation of GEP affects neuron survival and causes frontotemporal dementia. GEP has been identified as a therapeutic target from the global gene expression profiles of liver cancer. GEP has been shown to be up-regulated in liver cancer tissues and functional experiments have demonstrated that GEP controls proliferation, invasion and tumorigenicity. Thus, GEP is an important molecule for targeted therapy. Nonetheless, targeted therapy alone in clinical settings, in general, is not sufficient to eradicate solid tumors.

### SUMMARY

The following presents a simplified summary of the various embodiments in order to provide a basic understanding of some aspects described herein. This summary is not an extensive overview of the disclosed subject matter. It is intended to neither identify key or critical elements of the disclosed subject matter nor delineate the scope of the subject embodiments. Its sole purpose is to present some concepts of the disclosed subject matter in a simplified form as a prelude to the more detailed description that is presented later.

Various embodiments are directed to treating liver cancers exhibiting multidrug resistance (also referred to herein as chemoresistance). More specifically, the embodiments relate to targeting and/or suppressing a growth factor and a drug transporter to facilitate the treatment of chemoresistant liver cancer. The specific growth factor targeted can be granulin-epithelin precursor (GEP), which over-expresses in liver cancer cells and regulates proliferation, invasion and tumoriginicity. Suppression of GEP can enhance the apoptotic effect induced by chemotherapeutic agents, while up-regulation of GEP shows opposite trend. GEP has been shown to regulate drug transporters of the ATP-dependent binding cassette (ABC) drug efflux transporter family that play a role in chemoresistance, such as ABCB5 and ABCF1. Accordingly, the drug transporter targeted can be ABCB5 or ABCF1. These methods can further include applying chemotherapeutics in combination with targeting the growth factor and the drug transporter. Targeting the growth factor and drug transporter, in combination with chemotherapy can provide a treatment modality that can eradicate aggressive liver cancer cells.

According to an embodiment, methods are described for manipulating a growth factor (e.g., GEP) and drug transporters (e.g., ABCB5 or ABCF1) on a cell, as well as related products. In a further embodiment, described are methods for treating cancer cells using growth factor (e.g., GEP) and drug transporter (e.g., ABCB5 or ABCF1) binding molecules and suppression of growth factor and drug transporter molecules. Further described herein are sets of markers whose expression patterns can be used to differentiate different clinical conditions, such as high or low levels of a growth factor (e.g., GEP) and drug transporters (e.g., ABCB5 or ABCF1). Based on the different clinical conditions, a likelihood of cancer recurrence, drug sensitivity, and prognosis can be determined. Also described herein are methods of classifying and treating patients based on the prognosis are also provided herein.

The following description and the annexed drawings set forth in detail certain illustrative aspects of the disclosed subject matter. These aspects are indicative, however, of but a few of the various ways in which the principles of the various embodiments may be employed. The disclosed subject matter is intended to include all such aspects and their equivalents. Other advantages and distinctive features of the disclosed subject matter will become apparent from the following detailed description of the various embodiments when considered in conjunction with the drawings.

### BRIEF DESCRIPTION OF THE DRAWINGS

FIG. 1 shows GEP level regulated chemoresistance. (A) Hep3B cells were modulated for GEP levels: GEP suppression (−) and GEP overexpression (+). The transfectants were validated for GEP mRNA and protein level modulations (mean fluorescence intensity, MFI). (B) Positive correlation between GEP level and chemoresistance. GEP suppression demonstrated increased apoptotic populations and thus the cells were more sensitive to chemotherapeutic agents. GEP overexpression resulted in decreased apoptotic populations and thus the cells were more resistant to chemotherapeutic agents. GEP conferred chemoresistance to chemotherapeutic agents, including doxorubicin and cisplatin. (C) GEP modulated ABCB5 level. The liver cancer cells modulated for GEP levels were examined for ABCB5 expression. The change of GEP level conferred a moderate effect on the modulation of ABCB5 mRNA level, but a prominent effect on the regulation of ABCB5 protein level. The protein levels of GEP/ABCB5 (solid lines) were shown as mean fluorescence intensity (MFI) after subtraction for the corresponding isotype controls (dotted lines) for the flow overlay diagrams.

FIG. 2 shows increased ABCB5 in chemoresistant cells. (A) The chemoresistant cells demonstrated an increase of 10 to 16 folds in resistance to chemotherapeutic agents. The liver cancer cells were selected and expanded under different chemotherapeutic agents. These cells with “acquired resistance” were referred to as chemoresistant populations. The cells selected for resistance to doxorubicin were referred to as doxorubicin resistant cells. Similarly, the cells selected under cisplatin were referred to as cisplatin resistant cells. The drug IC50 values were determined by MTT assay. The doxorubicin resistant cells demonstrated an increase in resistance to doxorubicin by more than 16 folds compared to their parental cells (IC50 values were 1.78 and 0.11 μg/ml, respectively). The cisplatin resistant cells demonstrated an increase in resistance to cisplatin by more than 10 folds compared to their parental cells (IC50 values were 8.53 and 0.84 μg/ml, respectively). (B) ABCB5 up-regulation was observed in the chemoresistant cells.

FIG. 3 shows ABCB5 suppression enhanced doxorubicin uptake and cell apoptosis. (A) The cells were suppressed for ABCB5 expression by the siRNA approach. All of the cells showed decreased ABCB5 mRNA levels with siABCB5 (results of protein level suppression were shown in FIG. 4). (B) Doxorubicin content after 24 hours of doxorubicin (0.5 μg/ml) treatment. The majority of the Hep3B cells had doxorubicin uptake (76.4%) after 24 hours of doxorubicin incubation (solid line, compared to dotted line of the control). In contrast, GEP overexpressing cells and doxorubicin resistant cells had reduced populations with doxorubicin uptake (55.4% and 31.1%, respectively). Irrespective of the cells' baseline sensitivity to doxorubicin (middle panel), suppression of ABCB5 by the siRNA approach sensitized them to doxorubicin uptake (right panel). (C) Cell apoptosis after 24 hours of doxorubicin (0.5 μg/ml) treatment. Suppression of ABCB5 enhanced cell apoptosis in cells including the Hep3B, the GEP overexpression transfectants, and the doxorubicin-resistant cells. * P<0.05, ** P<0.01 vs. controls.

FIG. 4 shows characterizations of hepatic stem cells marker expressions in HCC cells. The double-positive subpopulation, GEP+ABCB5+ cells is shown in the upper right quadrant of the scatter plot at the left panel, gated in R2/The double-positive subpopulation gated in R2 was further distinguished for positivity of CD133 (scatter plot at the middle panel) and EpCAM (scatter plot at the right panel). (A) Hep3B cells. The majority of the ABCB5+ cells were also GEP+(28.0% cells). These GEP+ABCB5+ double-positive cells expressed CD133 and EpCAM. Suppression of ABCB5 by the siRNA approach effectively decreased ABCB5 expression, reduced the population of cells coexpressing GEP, and diminished the cell population expressing the hepatic stem cell markers CD133 and EpCAM. (B) GEP overexpression transfectants. Increased GEP expression level by transfection of GEP full-length cDNA increased the ABCB5+GEP+double positive population (64.6% compared to 28.0% in parental cells), and the majority of these cells were positive for CD133 and EpCAM. Suppression of ABCB5 expression by siRNA decreased the GEP+ABCB5 subpopulation, and reduced the cell population with hepatic stem cell markers CD133 and EpCAM. (C) Doxorubicin resistant cells. Increased ABCB5+GEP+ subpopulation (57.6% compared to 28.0% in parental cells) was observed in the chemoresistant cells, and these double-positive cells expressed the hepatic stem cell markers CD133 and EpCAM. Suppression of ABCB5 expression decreased the GEP+ABCB5+ subpopulation and the cell population expressing hepatic stem cell markers CD133 and EpCAM. * P<0.005, **P<0.001 vs. controls.

FIG. 5 shows decreased hepatic stem cell marker expression in liver cancer cells with suppression of ABCB5. Cells were examined by flow cytometry and mean fluorescence intensity (MFI) of each protein was shown. (A) Hep3B cells. (B) GEP overexpression transfectants. (C) Doxorubicin resistant cells.

FIG. 6 shows high recurrence rate of HCC with elevated GEP and ABCB5 expressions. (A) GEP overexpression was significantly up-regulated in HCC compared to the paralleled tumor-adjacent liver tissues (comprised with hepatitis and cirrhotic livers) and the normal livers from healthy individuals. (B) ABCB5 expression was elevated in HCC. The majority of normal livers from healthy individuals and tumor-adjacent livers from HCC patients showed undetectable ABCB5. (C) Kaplan-Meier recurrence-free survival plot according to GEP levels (log-rank test, P=0.028). There were 26 patients in the low GEP group and 36 patients in the high GEP group (median recurrence-free survival of 37.2 months and 8.0 months, respectively). (D) Kaplan-Meier recurrence-free survival plot according to ABCB5 levels (log-rank test, P=0.022). There were 36 patients with undetectable ABCB5 expression and 26 patients with ABCB5 expression (median recurrence-free survival of 32.4 months and 7.4 months, respectively).

TABLE 1 shows a Cox regression analysis for recurrence-free survival on gene expression and clinicopathological parameters.

TABLE 2 shows ATP-binding transporter genes. Genes whose expression level differed by at least two fold, in at least one sample, from their mean expression level across all samples were selected for further analysis. Leaving 7836 clones, there were 22 clones, 19 genes encoding the ATP-binding transporters. The genes were ranked according to the correlation coefficient values of their expression levels with GEP.

FIG. 7 shows microarray data in the first set of liver samples. GEP and ABCF1 expression levels were correlated (P<0.001).

FIG. 8 shows a validation of GEP and ABCF1 correlation (P<0.001). Independent sample set of liver samples were analyzed by real-time quantitative PCR.

FIG. 9 shows that ABCF1 mRNA levels in tumor tissue is significantly higher than the parallel non-tumor liver (P<0.001).

FIG. 10 shows a Kaplan-Meier plot on recurrence-free survival according to ABCF1 levels. Cells exhibiting a low concentration of ABCF1 exhibited a higher recurrence-free survival rate than cells exhibiting a high concentration of ABCF1 (log rank, P=0.001).

TABLE 3 shows a Cox regression analysis for recurrence-free survival on gene expression and clinicopathological parameters including ABCF1.

FIG. 11 shows effects of GEP antibody treatment and a chemodrug on enhancement of apoptosis in Hep3B.

FIG. 12 shows continuous monitoring of tumor size with GEP antibody treatment and a chemodrug. GEP antibody treatment in combination with a chemodrug showed improved growth inhibition compared to either treatment alone.

### DETAILED DESCRIPTION

Various aspects relate to the treatment of liver cancer. Traditional treatments involve curative surgery, such as a partial hepatectomy, and/or chemotherapy. Chemotherapy has marginal efficiency, and patients to exhibit poor survival outcomes. This can be due to a small portion of cells exhibiting multidrug resistance (also referred to as chemoresistance herein).

Multidrug resistance can result from a decreased intracellular drug accumulation, for example due at least in part to tumor cell expression of an ATP-dependent binding cassette (ABC) drug efflux transporter. The multidrug resistance phenotype in liver carcinogenesis is commonly mediated through overexpression of ABC drug transporters, including ABCB1 and ABCC1. These transporters enable liver cancer cells to efflux a broad range of chemically diverse chemotherapeutic agents.

Multidrug resistance can also be facilitated by tumorigenic stem cells. The contribution of tumorigenic stem cells to hematopoietic cancers has been established for some time. Although chemotherapeutic agents kill most of the tumor cells, a small population of cancer stem cells can be left behind. These remaining cancer stem cells can be protected from chemotherapy, for example, by ABCB5 and ABCF1.

Gene expression profiling studies of liver cancer have shown that hepatic cancer cells express GEP. GEP has been shown to be up-regulated in liver cancer tissues, and, functionally, GEP controls proliferation, invasion and tumorigenicity. Accordingly, GEP is an important molecule for targeted therapy. A therapeutic approach of GEP-targeted therapy for liver cancer using anti-GEP monoclonal antibody with an animal model has been previously demonstrated. However, targeted therapy alone in clinical settings is not sufficient to eradicate solid tumors.

As shown in the Experimental section, overexpression of GEP confers chemoresistance to liver cancer cells, while suppression of GEP renders the lover cancer cells chemosensitive. Additionally, GEP is the upstream from ATP-dependent binding cassette (ABC) drug efflux transporters (ABCB5 or ABCF1), so that GEP can regulate the protein level of ABCB5 and/or other drug efflux transporters, such as ABCF1. Suppression of ABCB5 or ABCF1 can render liver cancer cells chemosensitive. Targeting GEP and the drug efflux transporter, in combination with chemotherapy, can provide treatment modalities to eradicate aggressive, chemoresistant liver cancer cells.

Moreover, the GEP+ABCB5+ cells coexpressed the hepatic stem cell markers CD133 and EpCAM, and the sternness feature explained the high recurrence rate after curative partial hepatectomy for liver cancer that expressed GEP/ABCB5. Accordingly, GEP regulates chemoresistance through ABCB5, and GEP+ABCB5+ cells express hepatic stem cell markers.

Suppression of GEP and drug transporters is a viable treatment modality for liver cancer cells. Suppression of GEP and ABCB5 has been shown to increase uptake of chemotherapeutic by at least 20% when compared to suppressing GEP alone. In other embodiments, suppression of GEP and ABCB5 has been shown to increase uptake of chemotherapeutics by at least 40% when compared to suppressing GEP alone. Suppression of GEP and ABCB5 has been shown to increase the uptake of chemotherapeutic by at least 40% when compared to treatment with chemotherapeutics alone. Additionally, treatment suppressing GEP and drug transporters ABCB5 and ABCF1 increases chemotherapeutic by at least 45% when compared to treatment with chemotherapeutic alone. Additionally, treatment suppressing GEP and ABCB5 in combination with chemotherapy increases the apoptotic rate of liver cancer cells by at least 30%. In another embodiment, treatment suppressing GEP, ABCB5 and ABCF1 in combination with chemotherapy increases the apoptotic rate of liver cancer cells by at least 40%. Moreover, suppression of GEP and ABCB5 can reduce the number of drug resistant cancer stem cells by at least 25%. Suppression of GEP, ABCB5 and ABCF1 can reduce the number of drug resistant cancer stem cells by at least 35%.

Furthermore, treatment suppressing GEP and drug transporters in combination with chemotherapy increases the recurrence free survival time in at least 50% of patients by more than six months. According to a more preferred embodiment, treatment suppressing GEP and drug transporters in combination with chemotherapy increases the recurrence free survival time in at least 30% of patient by more than 12 months. According to a more preferred embodiment, treatment suppressing GEP and drug transporters in combination with chemotherapy increases the recurrence free survival time in at least 10% of patients by more than 24 months.

### EXPERIMENTAL

### Materials and Methods

### Clinical Specimens

The study protocol was approved by the Institutional Review Board of the University of Hong Kong/Hospital Authority Hong Kong West Cluster (HKU/HA HKW IRB). Between October 2002 and July 2005, 66 patients having curative partial hepatectomy for hepatocellular carcinoma (HCC) at Queen Mary Hospital, Hong Kong, were recruited with informed consent to the study. The same team of surgeons performed all the operations throughout this period. Clinicopathological data were prospectively collected. All patients had been diagnosed with primary HCC. Recurrence-free survival was the endpoint and was calculated from the date of surgery to the date of recurrence. Diagnosis of recurrence was based on typical imaging findings in contrast-enhanced computed tomography scan and an increase of serum alpha-fetoprotein level. In case of uncertainty, hepatic arteriography and post-Lipiodol computed tomography scan were performed, and, when necessary, fine-needle aspiration cytology was used for confirmation. Only 62 patients were included in the recurrence-free survival analysis. Four patients were excluded from the survival outcome analysis because of default follow-up, hospital mortality or concurrent radiofrequency ablation. Up to the date of analysis, the median follow-up time was 66.6 months.

### Cell Cultures and Assays

Human liver cancer cell lines, Hep3B and HepG2, were purchased from American Type Culture Collection (Manassas, Va.). Culture method has been previously described, for example by Ho J C, et al. Hepatology 2008; 47: 1524-1532 and Cheung S T, et al. Clin Cancer Res 2004; 10: 7629-7636. Stable transfectants for GEP overexpression and suppression have also been described. For chemoresistant populations, the Hep3B cells were plated out and selected under various chemotherapeutic agents of various concentrations at 10-fold dilution for 30 days. The highest dose that still had viable cells over the extended selection period was used and the cells expanded for further selection. The one-step selected cells were then plated out again and selected under escalating doses of the respective drug in a 2-fold manner for another 30 days. The two-step selection process could select a population of cells more resistant to chemotherapeutic agents. The cells selected for resistance to doxorubicin were referred to as doxorubicin resistant cells. Similarly, the cells selected under cisplatin were referred to as cisplatin resistant cells. The drug IC50 value was determined by MTT assay. For apoptosis assays, cells were incubated with or without chemotherapeutic agents for 24 to 48 hours. Apoptosis was determined by Annexin V-FITC (AV-FLI) and propidium iodide (PI-FL2) staining using flow cytometry. The total apoptotic population included the early apoptotic cells (high MFI of AV but low PI) plus the late apoptotic cells (high MFI of both AV and PI) of the scatter plot. The bar chart for apoptosis assay showed the net increase of apoptotic cells after designated time of treatment (e.g.=the apoptotic population under doxorubicin treatment for 24 hours minus the control with no doxorubicin). For doxorubicin uptake assays, cells were incubated with or without doxorubicin for 24 hours, and doxorubicin content (FL2) was analyzed by flow cytometry. Pilot studies had examined treatment time-points 0, 1, 3, 6 and 24 hours, and the latter three time-points had similar data profiles. Hence, treatment time-points 0, 1, and 24 hours were examined in the subsequent experiments for doxorubicin uptake and apoptosis assays. There was a time-dependent effect, and thus the data charts presented the data of the 24-hour treatment in comparison with the baseline data. Antibodies against ABCB5 (Everest Biotech Ltd, Oxfordshire, UK), CD133 (Miltenyi Biotec, Bergisch Gladbach, Germany), EpCAM (BD Biosciences, San Jose, Calif.) and GEP (described previously By Ho K C, et al. Hepatology 2008; 47: 1524-1532) were used in the immunofluorescence staining by flow cytometry (FACSCalibur, BD Biosciences).

### Real-Time Quantitative RT-PCR

Real-time quantitative RT-PCR was performed as described previously by Cheung S T, et al. Clin Cancer Res 2004; 10: 7629-7636. Quantification was performed with the ABI Prism 7700 sequence detection system (Applied Biosystems, Foster City, Calif.). Primers and probes for GEP have been described by Cheung S T, et al. Clin Cancer Res 2004; 10: 7629-7636. Primers and probe reagents for ABCB5 and control 18s were ready-made reagents (Pre-Developed TaqMan Assay Reagents, Applied Biosystems). The relative amount of GEP and ABCB5, which had been normalized with control 18s for RNA amount variation and calibrator for plate-to-plate variation, was presented as the relative fold change.

### RNA Interference

Three stealth small interfering RNAs (siRNA) specific to ABCB5 (HSS139171, HSS139172 and HSS139173) and a control siRNA with matched GC content were designed and synthesized by Invitrogen (Carlsbad, Calif.). Transfection was performed using Lipofectamine 2000 (Invitrogen) according to the instructions of the manufacturer. A total of 100 nmol/L of siRNA duplex was used for each transfection. Each set of transfection had three controls, including the cell plus Lipofectamine, the cell plus Lipofectamine and control siRNA, and the cell only control. These three controls had similar data profiles, and thus the data charts presented the average data of the controls. Comparison of the three siRNAs for ABCB5 indicated that HSS139172 had a more consistent effect on mRNA and protein suppression, and thus the data charts presented the average data of siABCB5 HSS139172.

### Statistical Analyses

All statistical analyses were performed by SPSS version 16.0 for Windows (SPSS Inc., Chicago, Ill.). Continuous variables were assessed by the Spearman correlation and compared between groups by Student's t-test. The GEP and ABCB5 mRNA levels were continuous variables, and the data were modeled as categorical variables in Kaplan-Meier and Cox regression analyses. The Youden index, i.e. sensitivity+specificity−I, was used to determine the optimal cutoff point for the prediction of 3-year recurrence-free survival. Other cutoff values, including the mean and the median, were also considered and examined. They were all able to segregate patients with similar clinical implications. The Youden index was employed to simultaneously maximize the sensitivity and the specificity of the prediction. The association of GEP, ABCB5 and tumor stage (AJCC tumor staging system) with recurrence-free survival was examined by univariate and multivariate Cox proportional hazards regression with a forward stepwise selection procedure. A P value less than 0.05 was considered statistically significant.

### Results

Growth Factor GEP Regulated Chemoresistance Through ABCB5

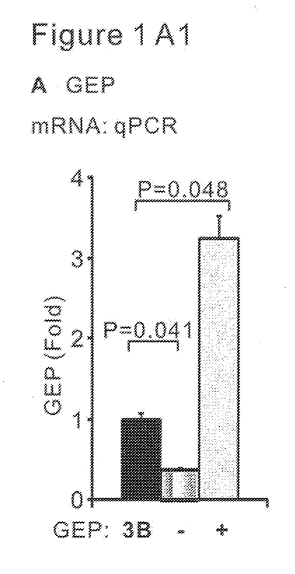

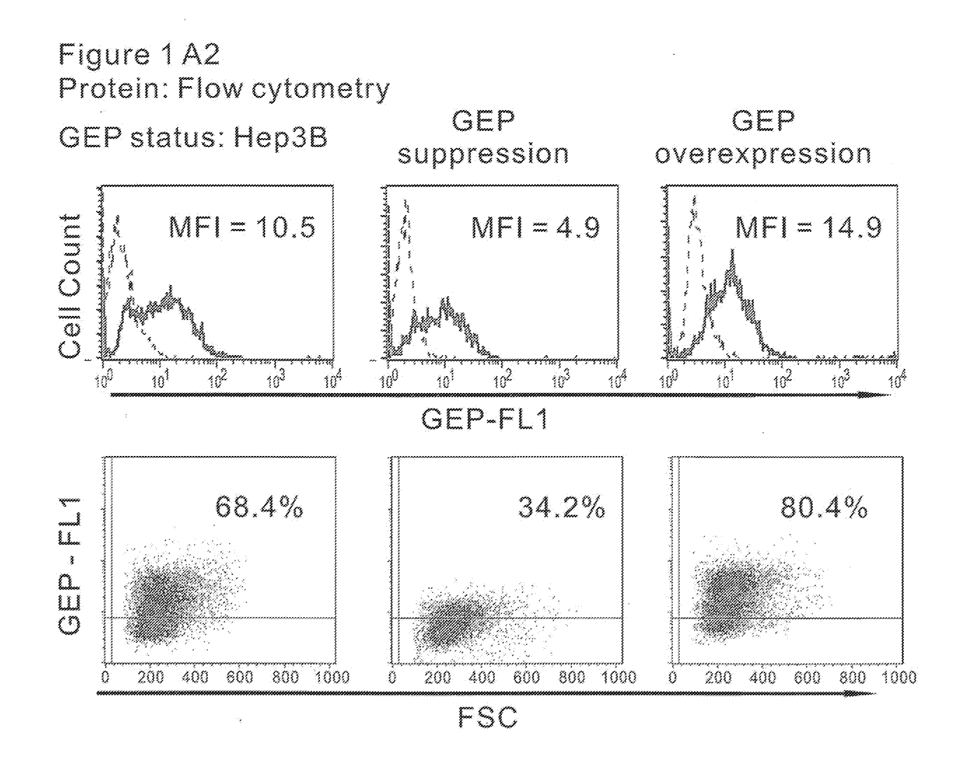

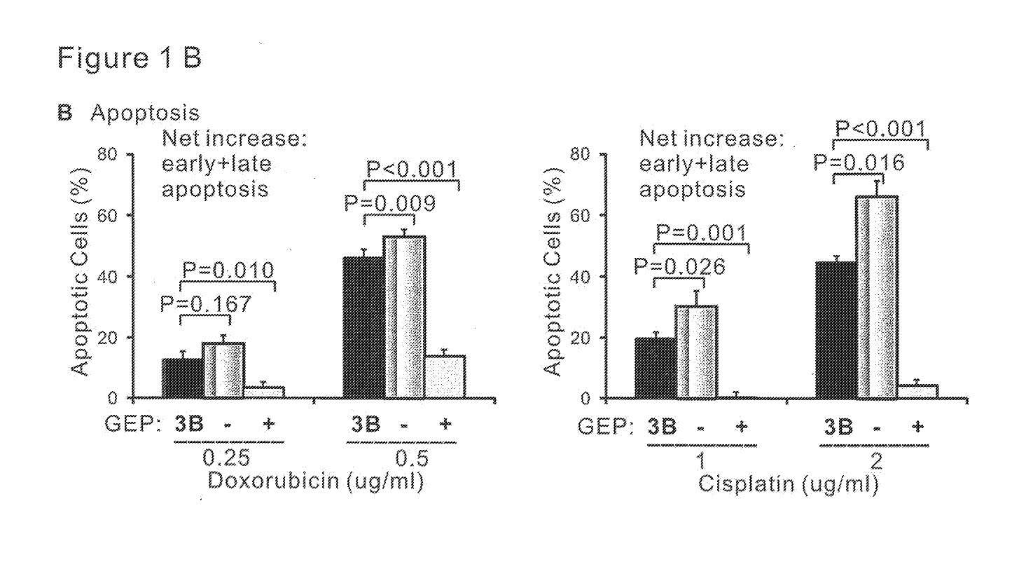

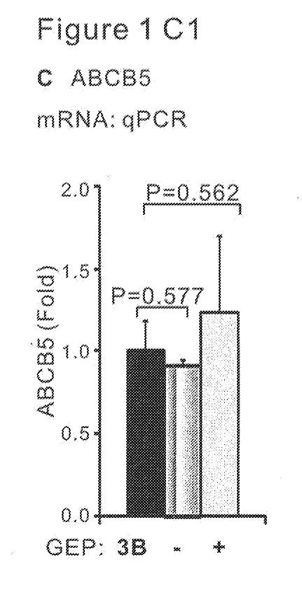

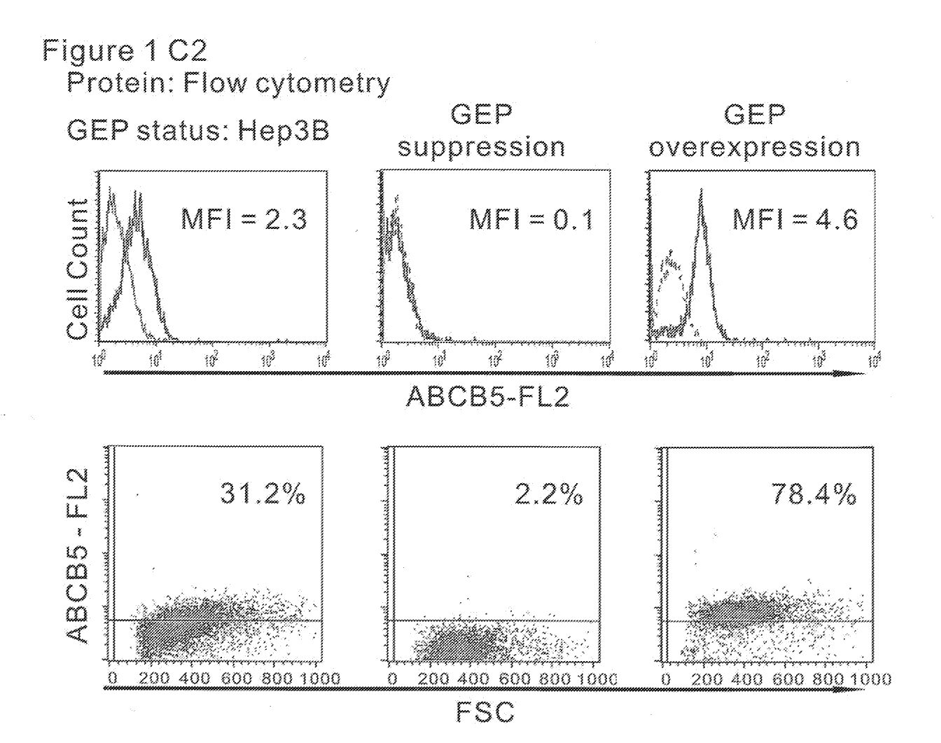

To examine whether GEP has a role in chemoresistance, transfection experiments were performed to overexpress or suppress GEP in HCC cells (FIG. 1 (A)). The transfectants were investigated for chemoresponses under doxorubicin and cisplatin treatments. We observed that suppression of GEP (−) sensitized the HCC cells to chemotherapy with enhanced apoptosis, while overexpression of GEP (+) rendered the HCC cells resistant to chemotherapeutic agents with fewer apoptotic cells (FIG. 1 (B)).

A number of ABC drug transporters were then screened to examine if GEP would regulate chemoresistance through modulating the drug transporter levels. GEP overexpression enhanced ABCB5 protein expression, while GEP suppression down-regulated ABCB5 protein expression (FIG. 1 (C)). Notably, the variation of GEP level demonstrated a prominent effect on the modulation of ABCB5 protein level. However, GEP level differences only moderately affected ABCB5 mRNA level in cell lines. Remarkably, the other common ABC drug transporters were not affected by GEP modulations, including ABCB1 (also named P-glycoprotein or MDRI), ABCCI (also named MRP1) and ABCC2 (also named MRP2) (data not shown).

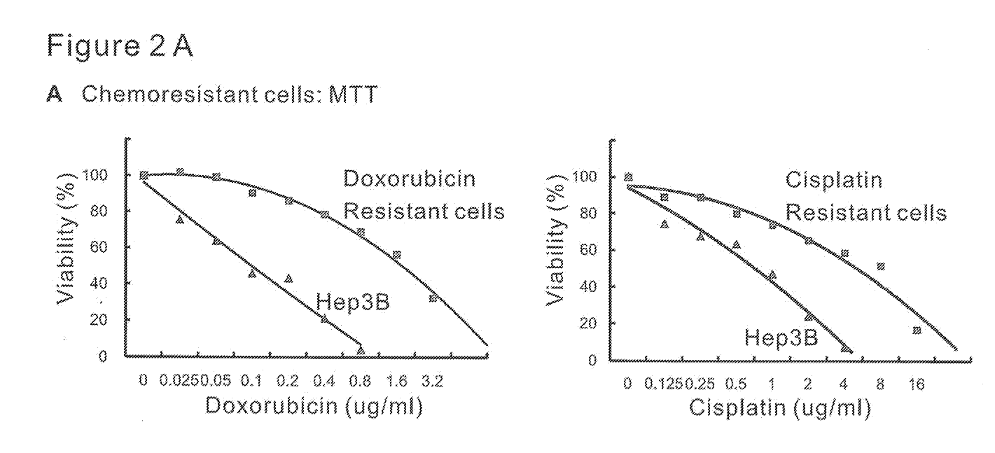

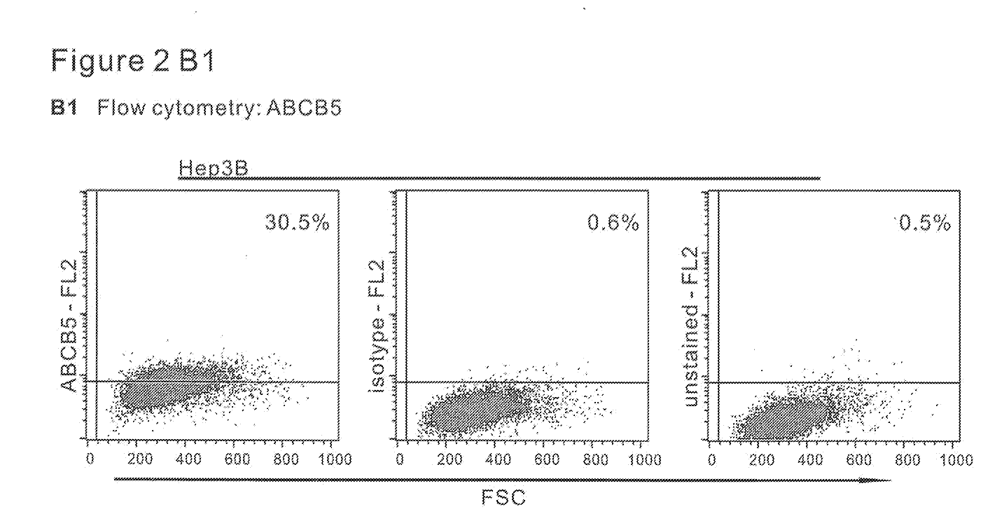

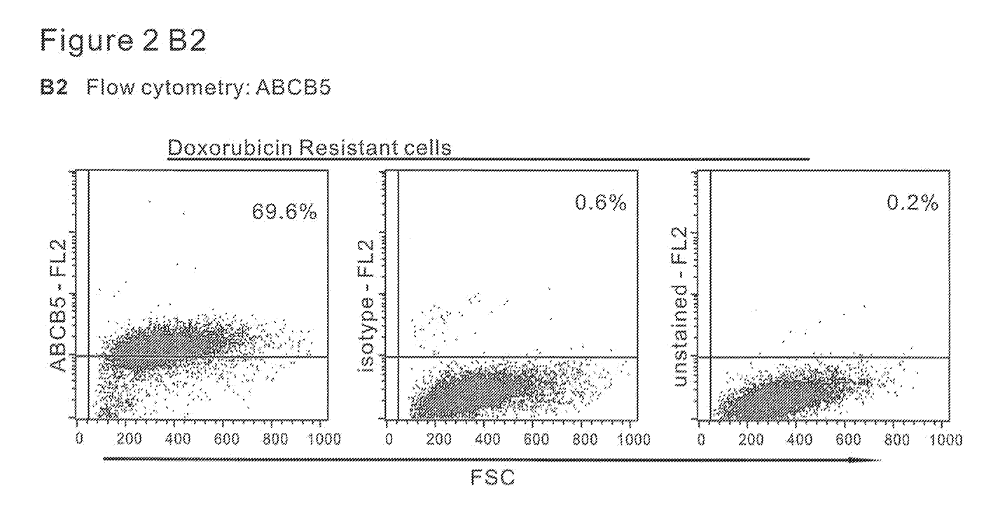

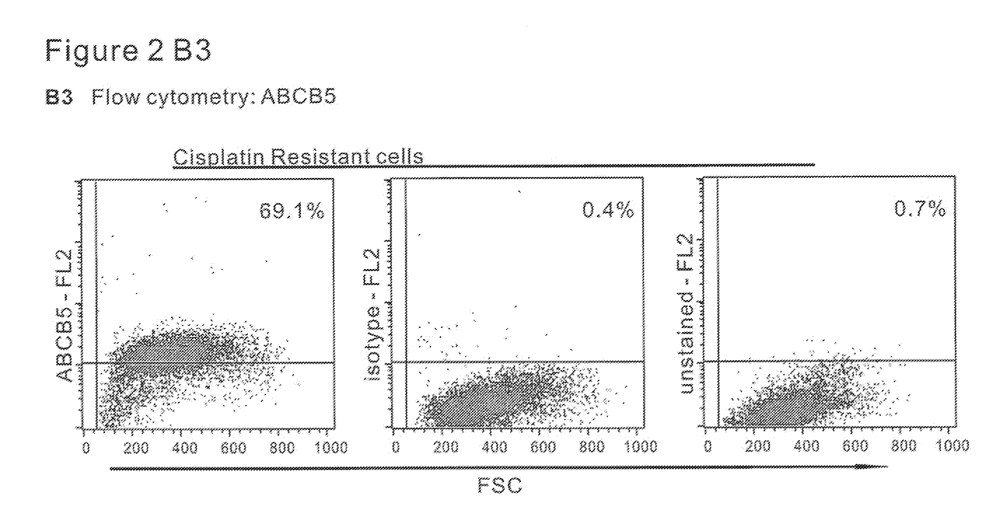

Chemoresistant HCC cells were used to examine the role of ABCB5. Cells were plated out and selected under different chemotherapeutic agents. The cells selected under doxorubicin were referred to as the doxorubicin resistant population, and they demonstrated increased resistance to doxorubicin compared to their parental cells (FIG. 2(A)). The cells selected under cisplatin were referred to as the cisplatin resistant population, and similarly they showed increased resistance to cisplatin. Both the doxorubicin and cisplatin resistant populations showed enhanced ABCB5 expression (FIG. 2(B)).

Cells with Elevated ABCB5 Reduced Doxorubicin Uptake

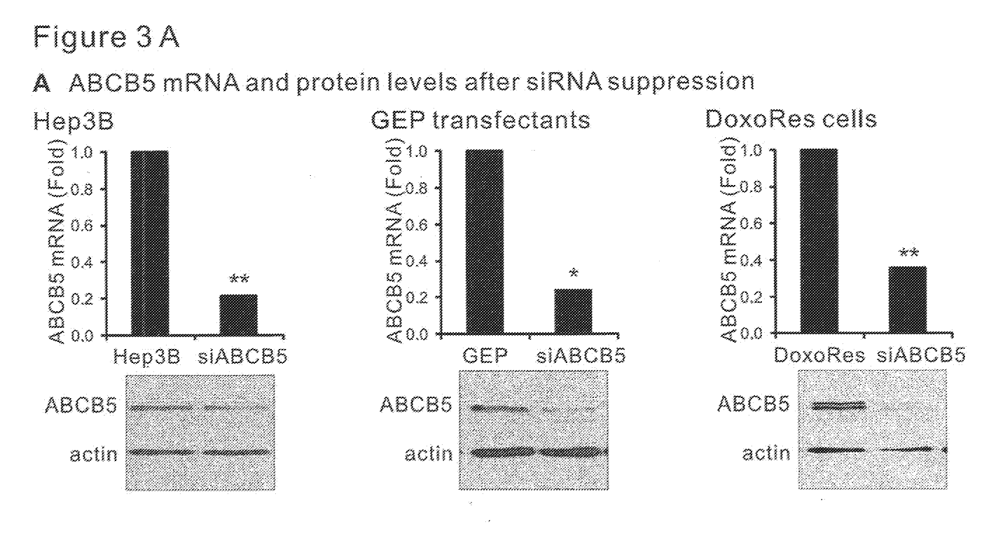

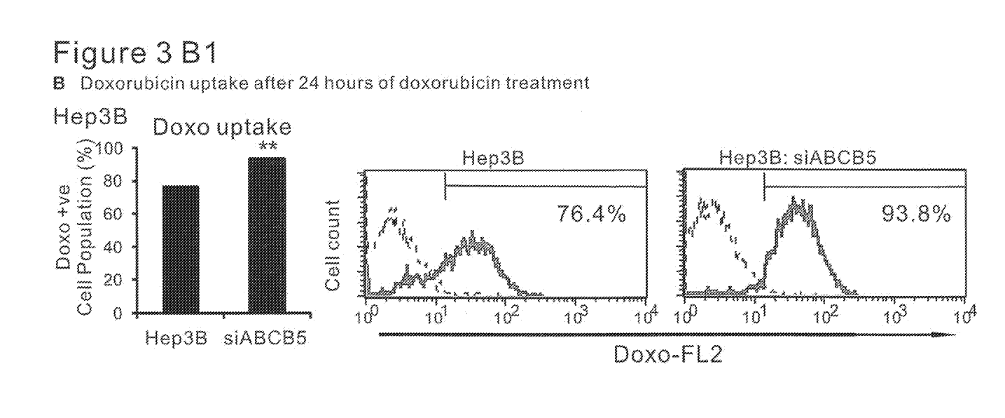

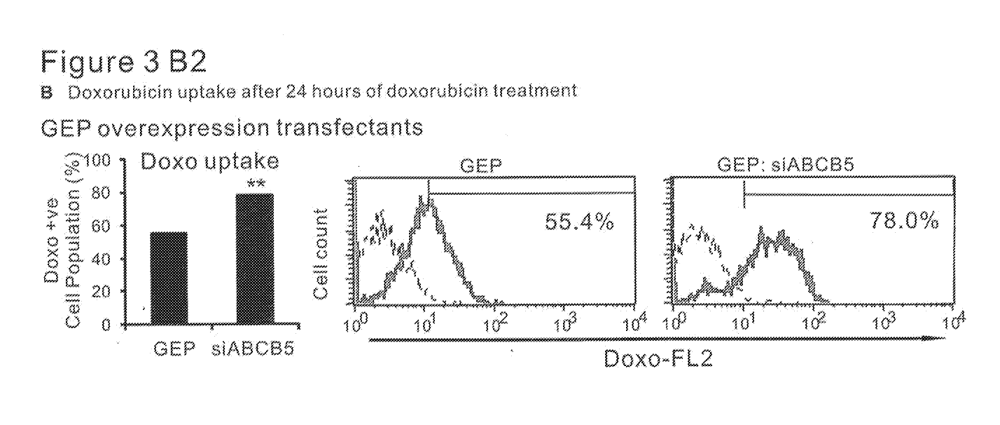

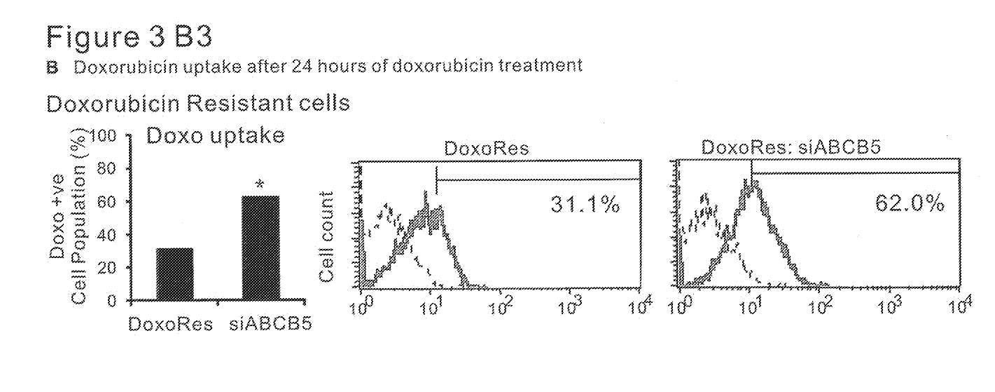

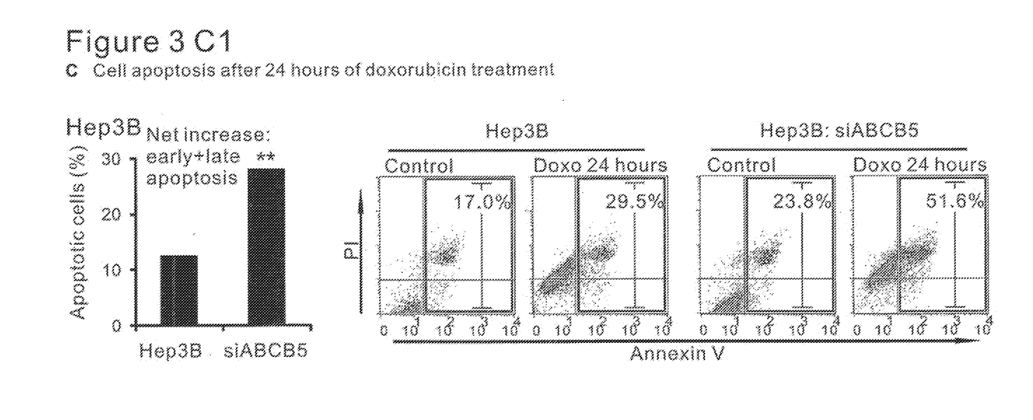

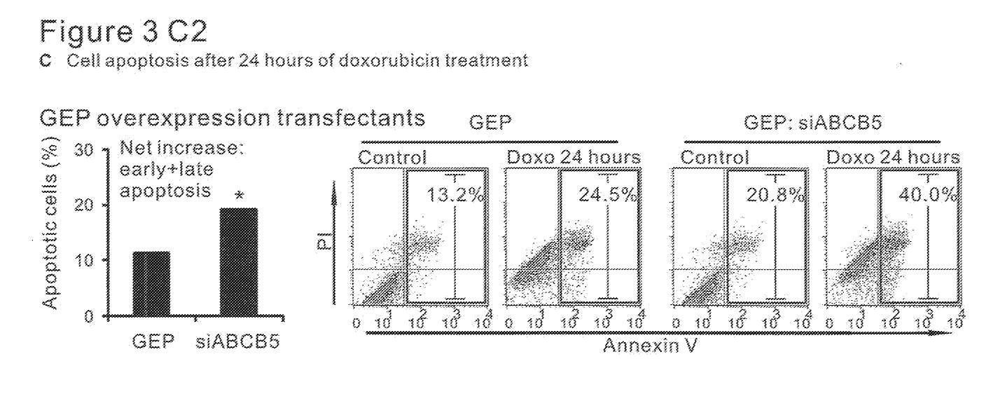

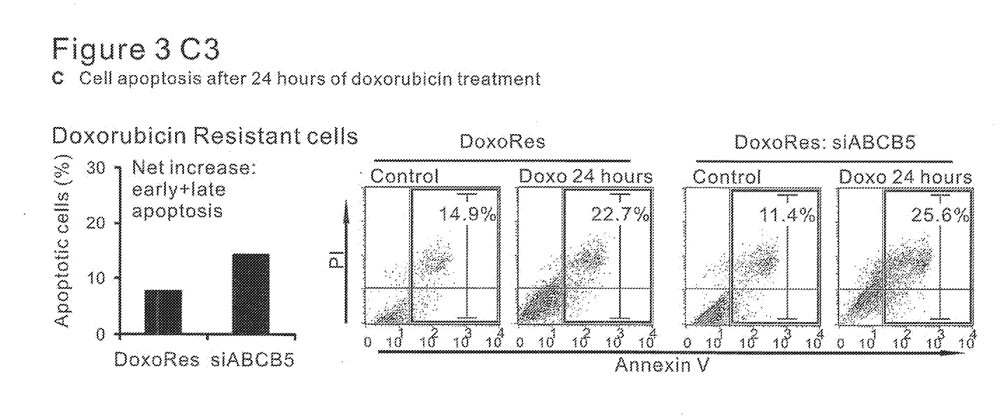

The different cell populations were exposed to doxorubicin and examined for drug uptake (FIG. 3). After doxorubicin treatment, GEP overexpression transfectants and doxorubicin resistant cells both demonstrated a lower doxorubicin uptake compared to the parental Hep3B cells (cell populations with doxorubicin were 55.4% and 31.1%, compared to 76.4% respectively). It was also noted that the Hep3B cells showed a lower ABCB5 level compared to GEP overexpression transfectants and doxorubicin resistant cells. Thus, the current data indicated that the ABCB5 level was negatively associated with doxorubicin uptake.

### Suppression of ABCB5 Sensitized the Cells to Doxorubicin Uptake and Apoptosis

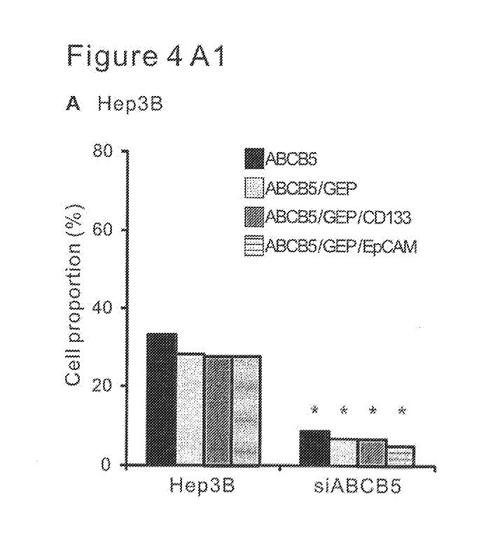

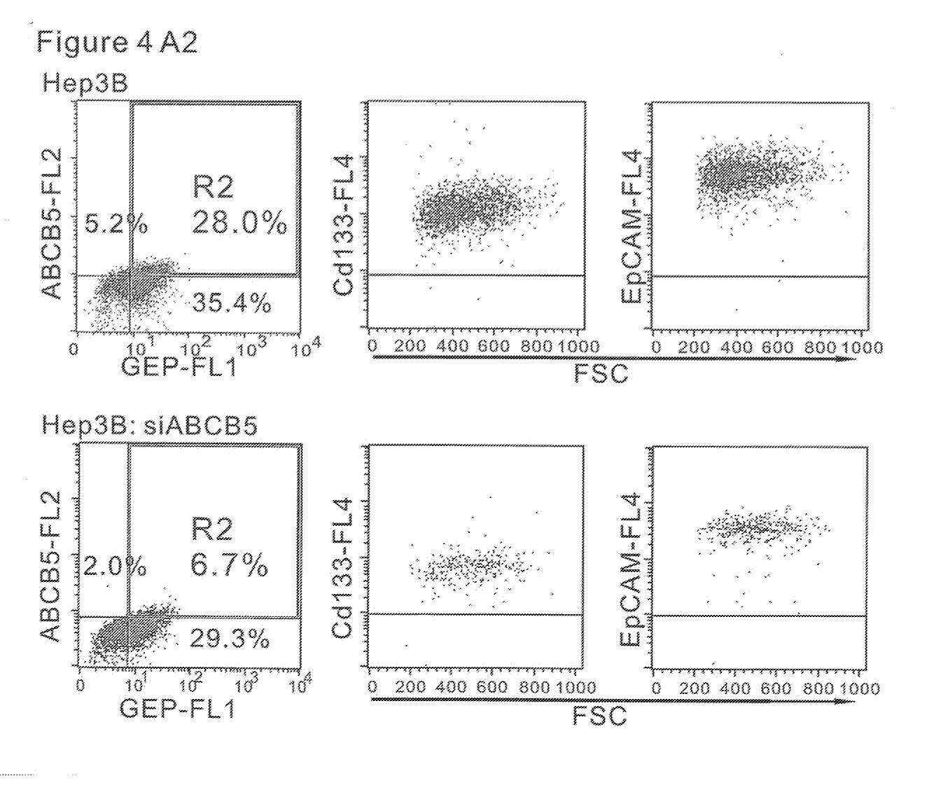

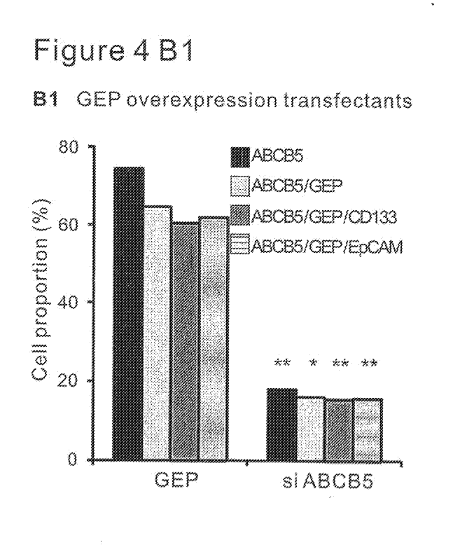

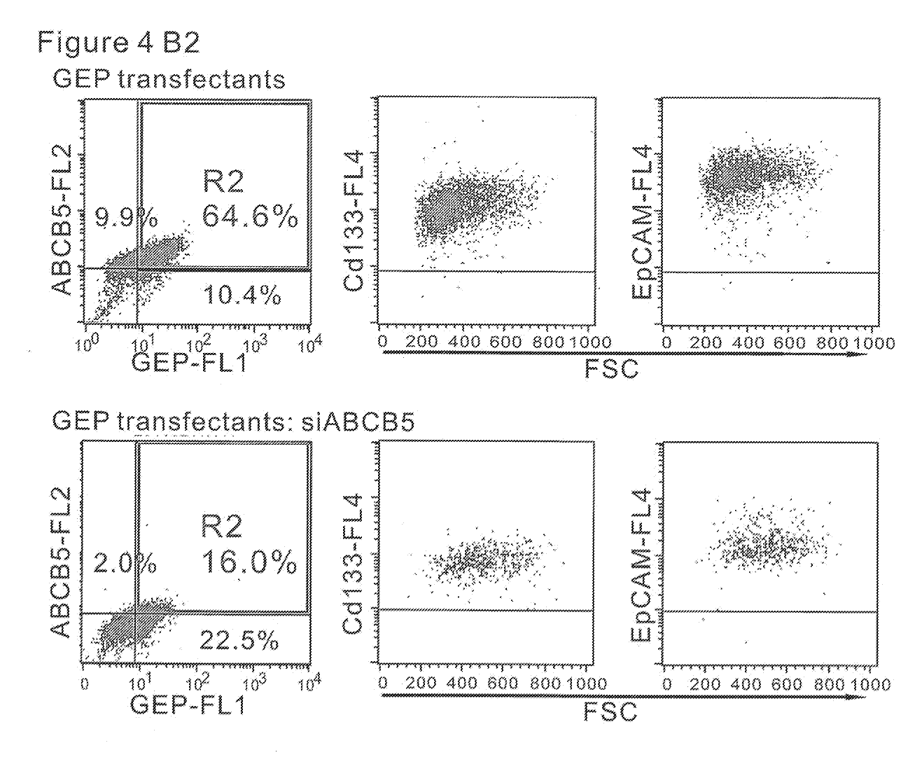

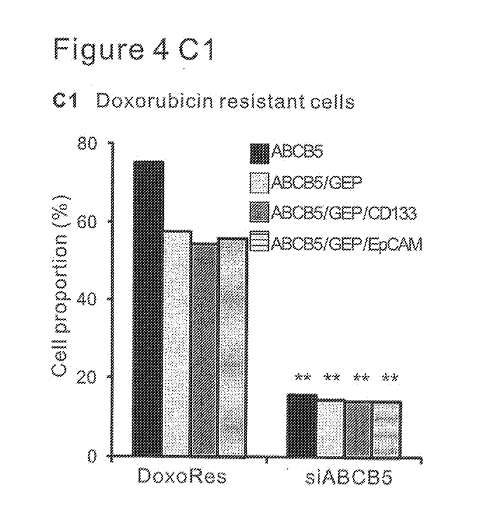

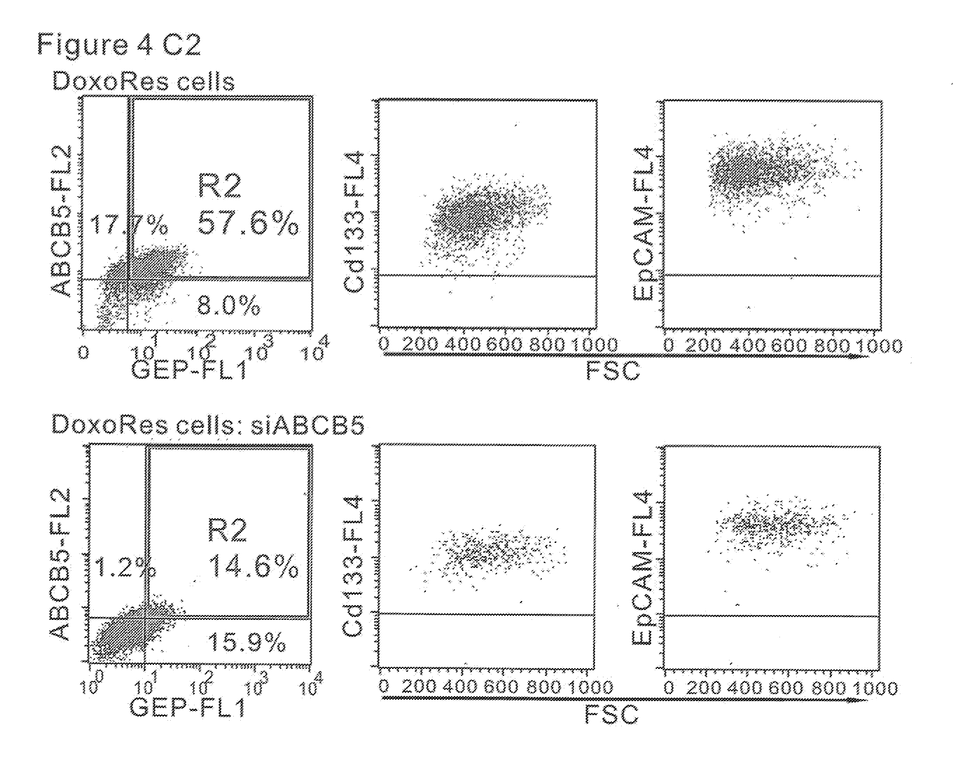

The results described earlier showed that GEP regulated chemoresistance, GEP regulated ABCB5 expression level, and ABCB5 demonstrated enhanced expression in chemoresistant populations. To consolidate if ABCB5 has a pivotal role in chemoresistance, ABCB5 expression levels were modulated by the siRNA approach and examined the functional effects. The Hep3B cells, GEP overexpression transfectants and doxorubicin resistant cells were transfected with three siRNAs against ABCB5. All the siRNAs were able to suppress ABCB5 mRNA and protein levels, and the siRNA that had a more consistent effect was shown (FIG. 3(A) on ABCB5 mRNA levels; FIG. 4 on ABCB5 protein levels).

In the Hep3B cells, suppression of ABCB5 demonstrated a significant increase in doxorubicin uptake (76.4% to 93.8% population with doxorubicin uptake) (FIG. 3B) and enhanced apoptosis (12.5% to 27.9% net increase in apoptotic populations) (FIG. 3C). It was further shown that ABCB5 suppression had a similar functional effect on liver cancer cells with a higher ABCB5 level, including the GEP overexpression transfectants and doxorubicin resistant cells. GEP overexpression transfectants demonstrated that ABCB5 suppression could enhance doxorubicin uptake (55.4% to 78.0%) and apoptosis (11.3% to 19.2%). Similarly, doxorubicin resistant cells showed that ABCB5 suppression could enhance doxorubicin uptake (31.1% to 62.0%) and apoptosis (7.7% to 14.2%). These data demonstrated that ABCB5 level regulated chemoresponse, and suppression of ABCB5 level could sensitize liver cancer cells to chemotherapeutic agents with increased intracellular drug content and enhanced apoptosis.

### GEP and ABCB5 Elevation in HCC

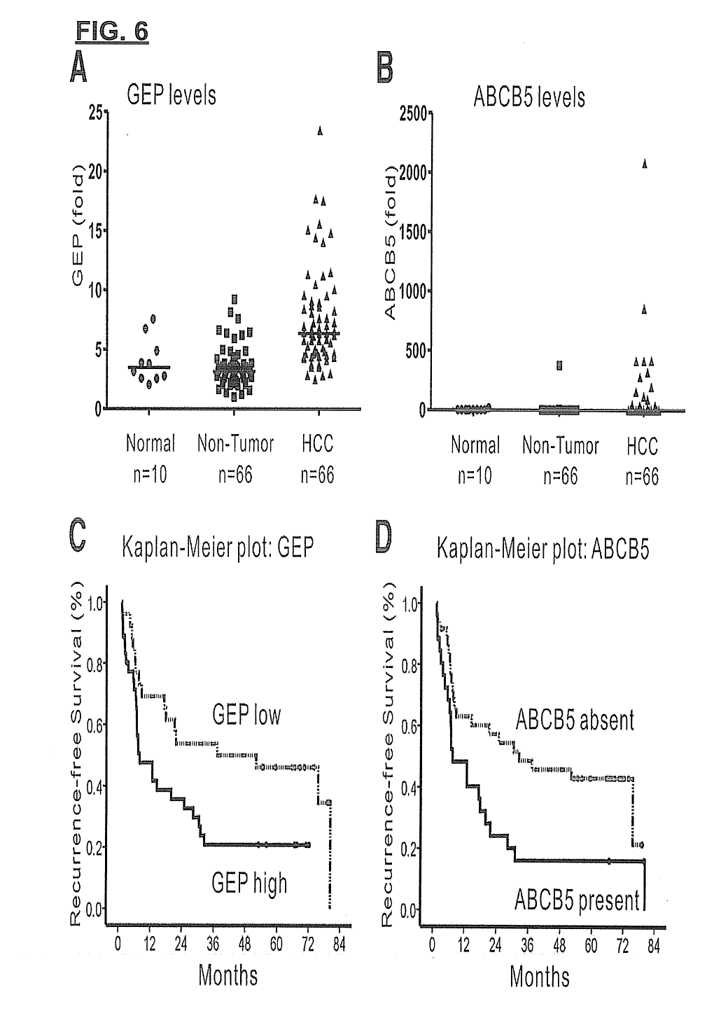

The association between ABCB5 was further examined with clinical specimens. GEP transcript and protein levels were reported in a previous study by Cheung S T, et al. Clin Cancer Res 2004; 10: 7629-7636. Herein, an independent patient cohort was recruited. Similar to the observation by Cheung, et al., GEP expression was significantly elevated in HCC compared to the paralleled tumor-adjacent liver tissues in the new sample set (Paired-Sample T-Test, P<0.001) and to the normal livers from healthy individuals (Independent Sample T-Test, P<0.001) (FIG. 6). This served as an independent study to demonstrate GEP is up-regulated in HCC in general. The tumor-adjacent liver tissues were comprised of hepatitis and cirrhotic livers, and the GEP expression level in these tissues was similar to that in the normal livers obtained from healthy individuals, indicating the uniqueness of GEP overexpression in HCC. ABCB5 was undetectable in the majority of the normal livers (90%, 9/10) and tumor-adjacent liver tissues (89.7%, 58/66). HCC tissues demonstrated elevated ABCB5 expression compared to the paralleled tumor-adjacent liver tissues (Paired-Sample T-Test, P=0.033) and to the normal livers from healthy individuals (Independent-Sample T-Test, P=0.022).

Gene expression levels were compared in the HCC samples, and the expression of GEP and that of ABCB5 significantly correlated (HCC n=66, Spearman's rho correlation coefficient=0.390, P=0.001). All the liver samples, including the tumor, tumor adjacent and normal liver tissues, were then included in the correlation analysis. Expressions of GEP and ABCB5 significantly correlated in the different types of liver samples investigated (n=142, Spearman's rho correlation coefficient=0.428, P=0.022). Accordingly, GEP and ABCB5 expressions significantly correlate in the clinical liver specimens, providing further evidence for the observation on cell models that GEP and ABCB5 were tightly associated.

Association of GEP and ABCB5 with Poor Prognosis

GEP protein expression has been shown to be associated with early intrahepatic recurrence by Cheung S T, et al. Clin Cancer Res 2004; 10: 7629-7636. The current patient cohort had extensive follow-up and thus the association between gene expression and recurrence-free survival was examined. Kaplan-Meier plot was used to examine patient outcome in association with gene expression. The patients were segregated into GEP low and GEP high groups with the Youden index maximized to determine the optimal cutoff value (FIG. 6(C), TABLE 1). There were 26 patients in the GEP low group (median recurrence-free survival 37.2 months) and 36 patients in the GEP high group (median recurrence-free survival 8.0 months). Patients with high GEP levels were found to have poor recurrence-free survival (log-rank test, P=0.028).

Prognosis analysis was performed based on ABCB5 expression. The optimal cutoff value for ABCB5 was determined by maximizing the Youden index, and the patients were segregated into ABCB5 absent and ABCB5 present groups (FIG. 6(D), TABLE 1). There were 36 patients in the ABCB5 absent group (median recurrence-free survival 32.4 months) and 26 patients in the ABCB5 present group (median recurrence-free survival 7.4 months). Patients with ABCB5 expression were shown to have poor recurrence-free survival (log-rank test, P=0.022).

To examine the prediction power for recurrence-free survival, Cox regression analysis was employed to compare the gene expression data and tumor stage (TABLE 1). By univariate Cox regression analysis, high GEP level [hazard ratio (HR)=2.3; 95% confidence interval (95% Cl)=1.2-4.6; P=0.016], ABCB5 expression (HR=2.3; 95% Cl 1.2-4.4; P 0.009) and advanced tumor stage (HR=2.7; 95% Cl=1.4-5.2; P=0.002) were significantly associated with poor recurrence-free survival. By multivariate Cox regression analysis, only ABCB5 expression (HR=2.1; 95% Cl=1.1-4.0; P=0.024) and advanced tumor stage (HR=2.5; 95% Cl=1.3-4.7; P=0.006) were found to be independent prognostic factors for recurrence-free survival. This part of the study showed that ABCB5 expression influenced the prognosis of liver cancer patients having curative partial hepatectomy.

### Expression of GEP/ABCB5 and Stem Cell Markers

Cancer stem cells have been known to express ABC drug transporters to protect themselves from chemotherapy. In addition, with the tumor bulk removed by curative partial hepatectomy, the high recurrence rate of liver cancer could be explained by the presence of cancer stem cells/tumor-initiating cells. The stem cell signature of hepatic cancer cells was further characterized. The majority of the GEP+ABCB5+ cells coexpressed hepatic cancer stem cell markers including CD133 and EpCAM in Hep3B cells (FIG. 4(A)). Increased GEP expression in the GEP overexpression transfectants increased the GEP+ABCB5+ population, and these cells coexpressed the stem cell markers (FIG. 4(B)). The doxorubicin resistant cells also showed an increased population of GEP+ABCB5+ with CD133 and EpCAM expressions (FIG. 4(C)).

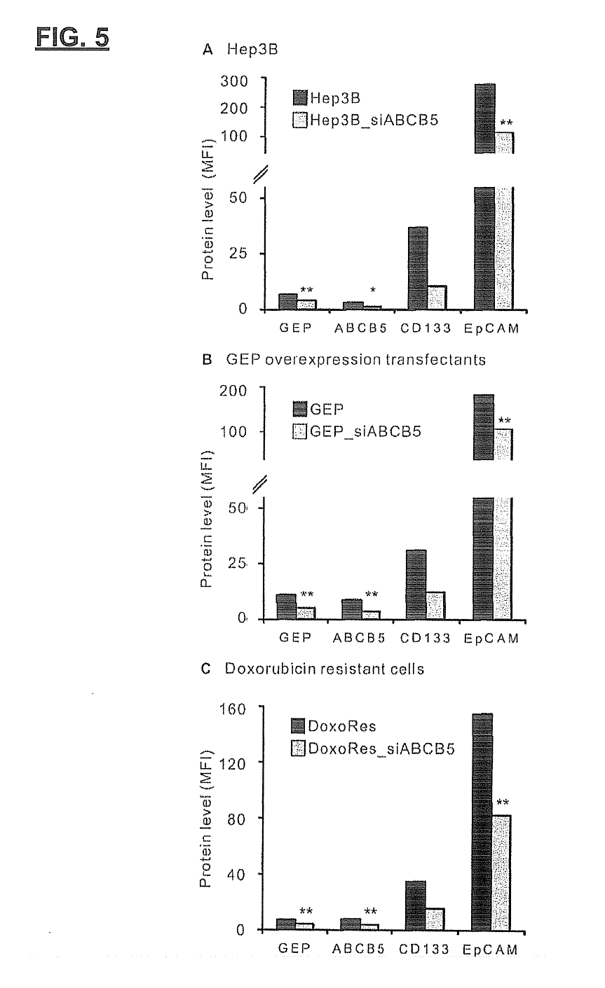

To further study the association between GEP/ABCB5 and stem cell properties, the cells were then suppressed for ABCB5 by the siRNA approach. All the cells, including Hep3B, GEP overexpression transfectants and doxorubicin resistant cells, demonstrated decreased ABCB5 expression. Notably, the population of GEP+ABCB5+ cells was decreased by siABCB5, and the majority of these double-positive cells coexpressed the hepatic cancer stem cell markers CD 133 and EpCAM. Importantly, suppression of ABCB5 not only decreased the triple positive cell populations (FIG. 4) but also the overall populations with hepatic stem cell markers CD133 and EpCAM expressions (FIG. 5). Thus, the GEP+ABCB5+ population was highly associated with the hepatic cancer stem cell population. The data supported the observation that GEP+/ABCB5+ HCC was associated with increased cancer recurrence after curative surgery.

### GEP Regulation of Other Drug Transporters

Since only 45% of HCC shows detectable ABCB5, it was hypothesized that GEP may regulate other drug transporters in addition to ABCB5. Liver cancer gene expression profiles were re-examined, and, as shown in TABLE 2, the ATP-binding transporters were ranked by the correlation coefficient values of their expression levels with GEP. The microarray data were validated in an independent sample set and independent research approach real-time quantitative RT-PCR.

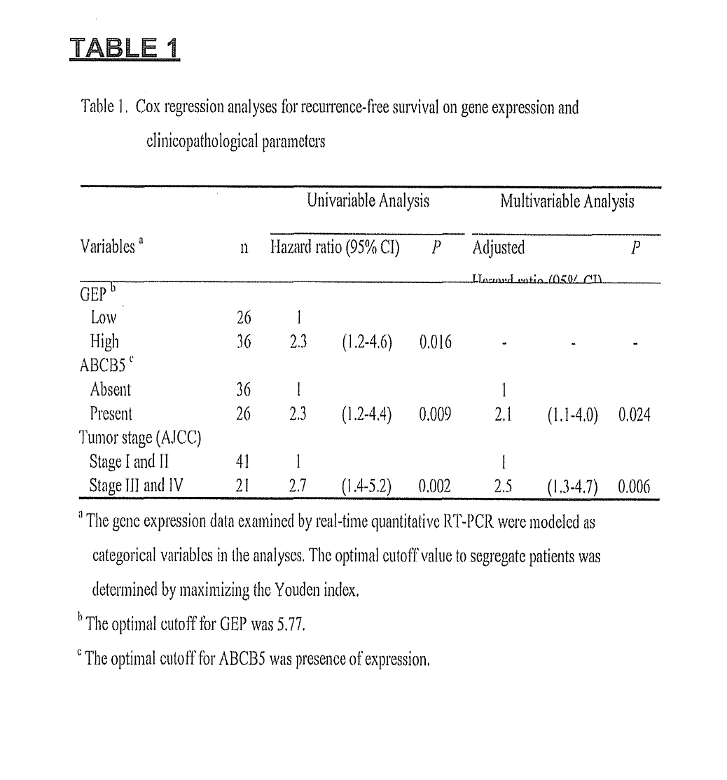

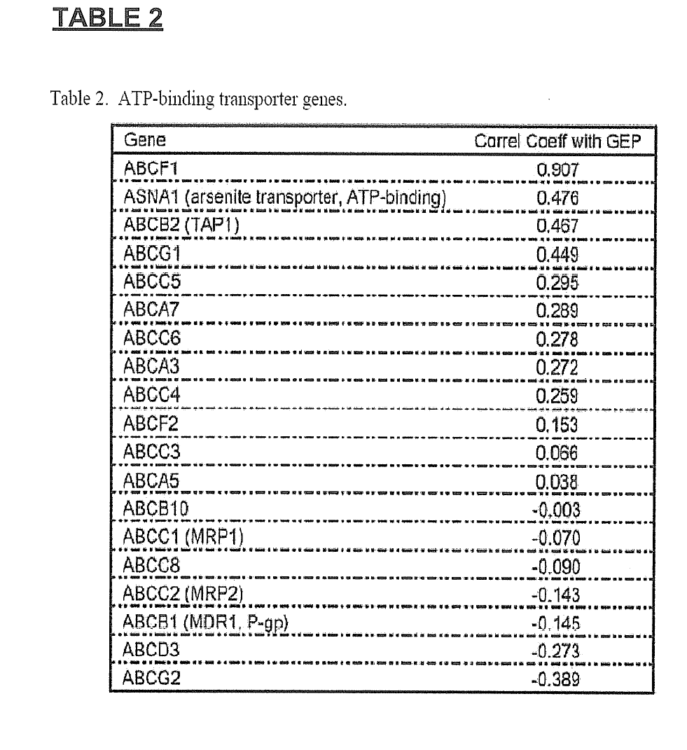

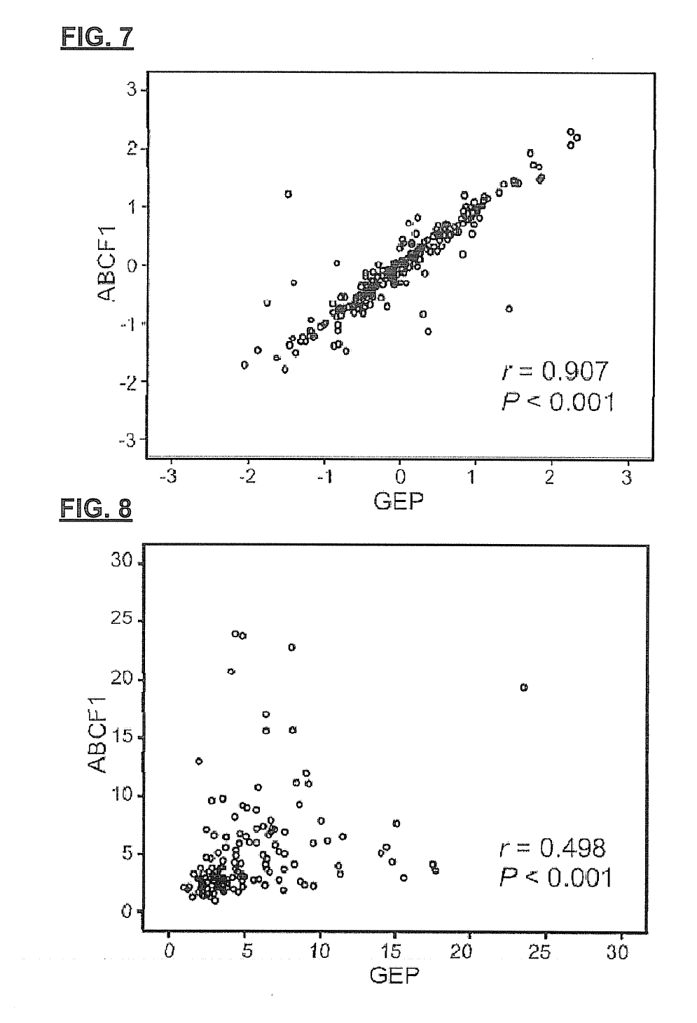

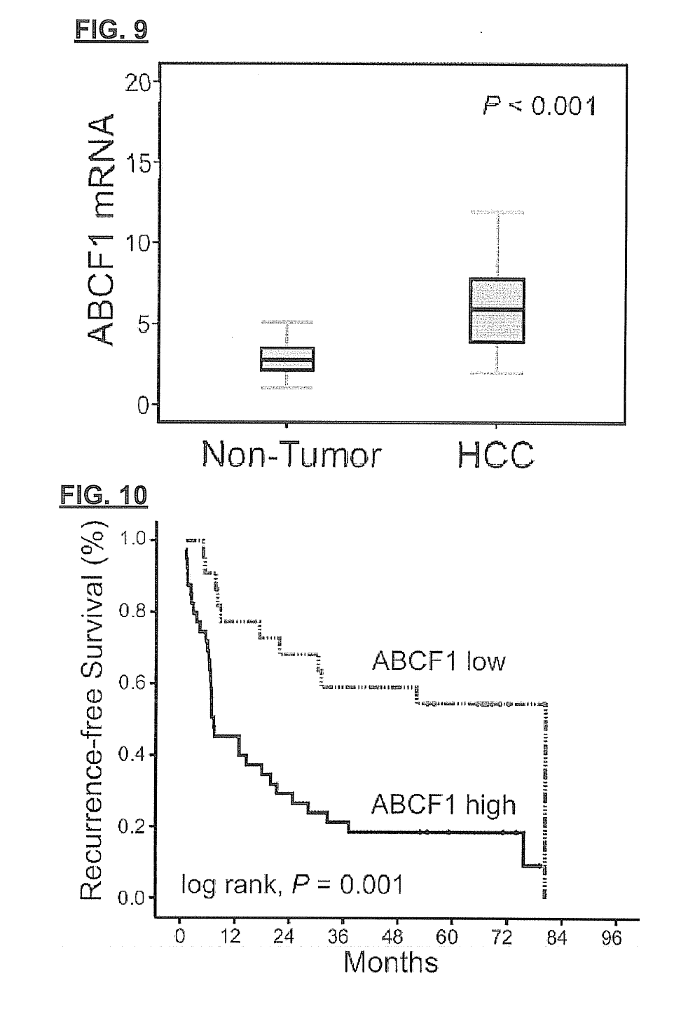

One such ATP-binding transporter is ABCF1. GEP and ABCF1 expression levels were significantly correlated (P<0.001) (FIGS. 6-7). ABCF1 expression was up-regulated in the tumor as compared with the adjacent non-tumor liver (P<0.001) (FIG. 9). The increased ABCF1 expressions were associated with poor recurrence-free survival (log-rank test, P=0.001) (FIG. 9, TABLE 3).

GEP Antibody in Combination with Chemodrug

Liver cancer Hep3B cells received different cell assay treatments. Control received no treatment. A23 received treatment with 100 μg/ml GEP antibody A23. Cis received a treatment of 4 μg/ml of the chemotherapeutic cisplatin. Cis+A23 received a combination treatment of GEP antibody A23 (100 μg/ml) plus cisplatin (4 μg/ml). Cells were harvested, stained with Annexin V (AV) and propidium iodine (PI), then flow analysis. The total apoptotic population included the early apoptotic cells (high mean fluorescence intensity of AV but low PI) plus the late apoptotic cells (high mean fluorescence intensity of both AV and PI). GEP antibody treatment in combination with chemodrug enhanced cancer cell apoptosis compared to chemodrug alone (FIG. 11).

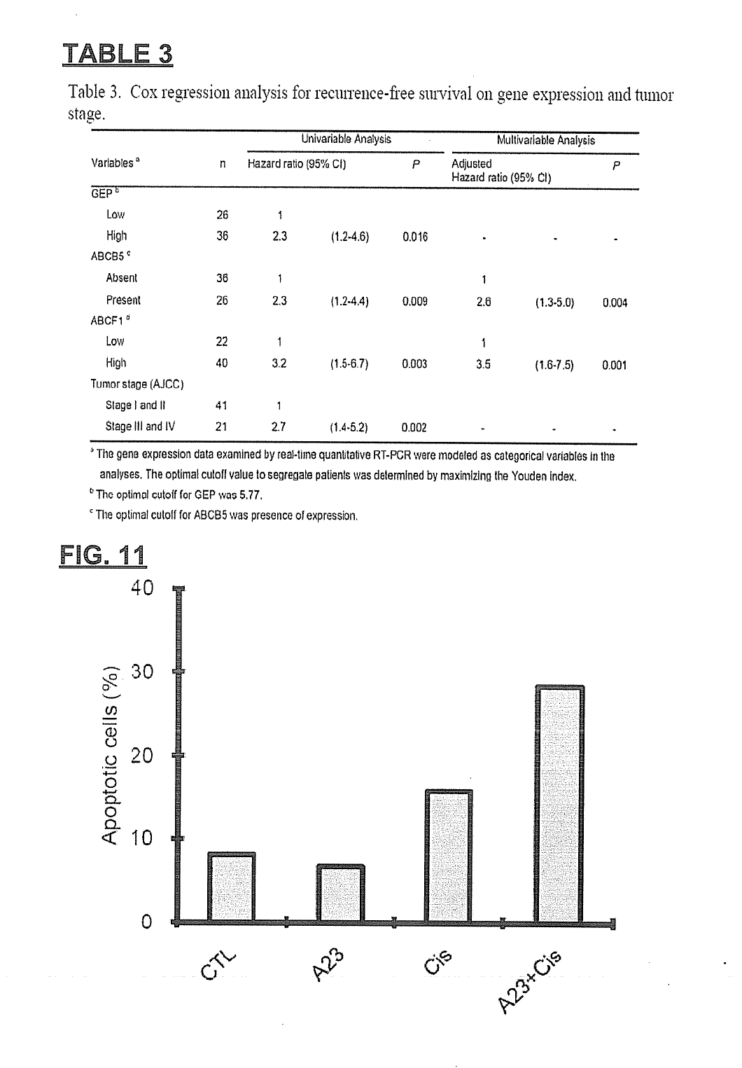

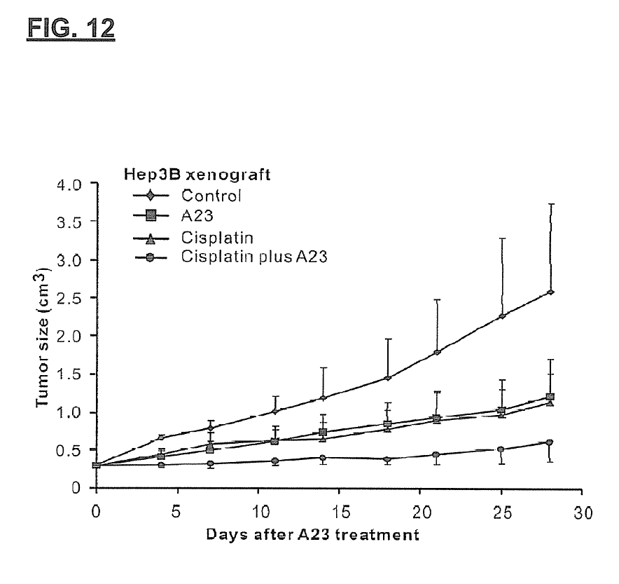

In an animal model, Hep3B cells were injected subcutaneously into nude mice and tumor growth was allowed to 0.3 cm³. Tumors were treated for one month with intra-peritoneal injection of GEP antibody A23 (0.1 mg twice per week), or cisplatin (0.1 mg, once per week), or a combination of A23 plus cisplatin, or control saline. Nude mice body weight 20-25 gm. Tumor size was continuously monitored. Tumor size was calculated according to the formula AB²/2, where A and B were the largest and smallest dimensions, respectively. GEP antibody treatment in combination with chemodrug showed improved growth inhibition compared to either treatment alone (FIG. 12).

### Discussion

### Biological Functions of GEP and ABCB5

GEP is a growth factor involved in tumorigenesis of human cancers of the prostate, bladder, ovary, and breast. GEP has been implicated in murine fetal development and wound response. Furthermore, GEP promotes neuronal cell survival, and mutation of GEP causes frontotemporal dementia. Thus, GEP is an important growth factor involved in many physiological situations. GEP regulates proliferation, invasion and tumorigenesis of liver cancer. Neutralization of GEP by the antibody approach inhibits tumor growth.

In the current study, we observed that overexpression of GEP conferred liver cancer cells chemoresistance while suppression of GEP rendered the cells chemosensitive. In addition, GEP modulated ABCB5 protein level, and suppression of ABCB5 level rendered the liver cancer cells chemosensitive. Furthermore, the GEP+ABCB5+ cells coexpressed the hepatic stem cell markers CD133 and EpCAM, and the sternness feature explained the high recurrence rate after curative partial hepatectomy for liver cancer that expressed GEP/ABCB5. This is the first study to demonstrate that GEP regulates chemoresistance through ABCB5, and that GEP+ABCB5+ cells express hepatic stem cell markers.

### Drug Resistance

Increasing evidence had revealed the role of GEP in mediating resistance to a number of clinical drugs in a variety of cancer types. Overexpression of GEP had been shown to render breast cancer cells resistant to tamoxifen and trastuzumab, multiple myeloma cells insensitive to dexamethasone, and ovarian cells resistant to cisplatin. However, the exact signaling whereby GEP confers drug-resistance had not been elucidated and whether GEP regulates drug transporters was not known from the literature. In this study, GEP was discovered to have a prominent effect in modulating ABCB5 protein level (FIG. 1 (C)). Thus, GEP has a positive effect in stabilizing ABCB5 protein or affecting the ABCB5 translation rate. In addition, suppression of ABCB5 by the siRNA approach resulted in reduced GEP protein levels (FIG. 4) but had no significant effect on GEP transcript levels (data not shown). The observation further supports that GEP protein and ABCB5 protein are able to stabilize each other.

### Cancer Stem Cells and Drug Resistance

ABCB5+ melanoma cells were known to be capable of self-renewal and differentiation and to possess greater tumorigenic capacity compared to the ABCB5− subpopulation. ABCB5 was known to express in CD133+ progenitor cells of melanocytes and mediate resistance to doxorubicin. Furthermore, the ABCB5+ subpopulation are known to have T-cell modulatory functions that may allow the subpopulation to evade host antitumor immunity. However, the signaling molecule that regulates ABCB5 protein level was previous unknown. This study demonstrates that GEP modulates ABCB5 protein level, and that enhanced GEP increased ABCB5 protein level while suppression of GEP decreased ABCB5 protein level. Importantly, suppression of either GEP or ABCB5 sensitized the cancer cells to chemotherapeutic agents.

Accordingly, chemoresistance and poor survival outcome are dictated by a subset of GEP+ABCB5+ liver cancer cells. Targeting the specific growth factor/drug transporter, in combination with chemotherapy, can provide treatment modalities to eradicate the aggressive liver cancer cells.

### GEP Regulation of Other Drug Transporters

To elucidate the signaling mechanism of how GEP regulates chemo-resistance, a number of common ATP-dependent binding cassette (ABC) drug efflux transporters reported in the literature have been examined. GEP was shown to modulate the expression of the drug transporter ABCB5, and blockage of ABCB5 sensitized the liver cancer cells to chemotherapeutic agents and attenuated the expression of hepatic cancer stem cell markers CD133 and EpCAM. Furthermore, GEP and ABCB5 expression levels were significantly correlated in clinical samples, and were associated with recurrence of hepatocellular carcinoma after partial hepatectomy. GEP controls growth, regulates chemo-resistance through the drug transporter ABCB5 and hepatic cancer stem cell marker expressions, partly explaining the rapid recurrence after tumor resection and features associated with chemo-resistance in liver cancer.

The current study reports the genomic approach to systematically examine GEP-associated genes in relation to chemo-resistance. Notably, GEP expression is detectable in all liver cancer tissues while only 45% show detectable ABCB5 transcript. Thus, GEP regulates chemo-resistance through ABCB5 only in a subset of liver cancer. Therefore, GEP may regulate other drug transporters in addition to ABCB5.

The liver cancer gene expression was re-examined, and the ABC drug transporter family members were ranked in association with GEP expression patterns (TABLE 2). The ABC genes that have shown high correlation with GEP expressions in the microarray hybridization datasets were further validated in an independent cohort of clinical specimens using the independent research platform real-time quantitative RT-PCR. The expression levels of drug transporter ABCF1 were significantly up-regulated in the tumor as compared with the adjacent non-tumor liver (P<0.001), and that the increased expressions were associated with poor disease-free survival (log-rank test, P=0.001). In summary, chemo-resistance and poor survival outcome are dictated by a subset of GEP+ABC+ liver cancer cells. Targeting the specific growth factor/drug transporter (e.g., ABCB5 or ABCF1), in combination with chemotherapy, could provide treatment modalities to eradicate aggressive liver cancer cells.

GEP Antibody in Combination with Chemodrug

Treatment of liver cancer with a combination of GEP antibody A23 and a chemodrug exhibited greater apoptotic effect than either the GEP antibody A23 or the chemodrug alone. Accordingly, targeting the specific growth factor in combination with chemotherapy can provide a more effective treatment modality to eradicate aggressive liver cancer cells.

With respect to any figure or numerical range for a given characteristic, a figure or a parameter from one range may be combined with another figure or a parameter from a different range for the same characteristic to generate a numerical range.

Other than in the operating examples, or where otherwise indicated, all numbers, values and/or expressions referring to quantities of ingredients, reaction conditions, etc., used in the specification and claims are to be understood as modified in all instances by the term “about.”

The embodiments as disclosed and described in the application are intended to be illustrative and explanatory, and not limiting. Modifications and variations of the disclosed embodiments, for example, of the processes and apparatuses employed (or to be employed) as well as of the compositions and treatments used (or to be used), are possible; all such modifications and variations are intended to be within the scope of this application.

What is claimed is:

1. A method for treating hepatic cancer cells, comprising:
administering a treatment comprising a granulin-epithelin precursor (GEP) antibody and a chemodrug to a hepatic cancer cell;
suppressing granulin-epithelin precursor (GEP);
regulating expression of a drug transporter downstream from GEP through the suppressing GEP; and
sensitizing the hepatic cancer cell to the chemodrug.

2. The method of claim 1, wherein the drug transporter is an ATP-dependent binding cassette (ABC) drug efflux transporter.

3. The method of claim 2, wherein the drug transporter is ABCB5.

4. The method of claim 2, wherein the drug transporter is ABCF1.

5. The method of claim 1, wherein the administering the treatment further comprises administering the treatment comprising the GEP antibody, a drug transporter-specific antibody and the chemodrug to the hepatic cancer cell.

6. The method of claim 1, wherein the sensitizing the hepatic cancer cell to the chemodrug further comprises enhancing apoptosis of the chemodrug on the hepatic cancer cells by at least 20%.

7. The method of claim 1, wherein the sensitizing the hepatic cancer cell to the chemodrug further comprises enhancing apoptosis of the chemodrug on the hepatic cancer cells by at least 30%.

8. A method for delivering a therapeutic agent to a hepatic cancer cell, comprising:
contacting a hepatic cancer cell with a molecule that selectively binds to granulin-epithelin precursor (GEP) conjugated to a chemotherapeutic agent in an effective amount to deliver the chemotherapeutic agent to an intracellular compartment of the hepatic cancer cell.

9. The method of claim 8, further comprising:
suppressing GEP; and
down-regulating expression of a drug transporter downstream from the GEP.

10. The method of claim 9, further comprising sensitizing the hepatic cancer cell to the chemotherapeutic agent and increasing a rate of apoptosis of the hepatic cancer cell by at least 20%

11. The method of claim 9, further comprising increasing uptake of the chemotherapeutic by at least 40% compared to administering the chemotherapeutic alone.

12. The method of claim 9, wherein the drug transporter is ABCB5.

13. The method of claim 9, wherein the drug transporter is ABCF1.

14. The method of claim 9, further comprising increasing an apoptotic rate of the cancer cell by at least 30%

15. A composition that increases an apoptotic effect of a chemodrug, comprising:
a granulin-epithelin precursor (GEP) antibody that selectively binds to GEP;
a therapeutic agent, comprising the chemodrug,
wherein the GEP antibody is co-formulated with the therapeutic agent.

16. The composition of claim 15, wherein the GEP antibody selectively binds to GEP and thereby promotes down-regulation of a drug transporter protein.

17. The composition of claim 16, wherein the drug transporter is ABCB5.

18. The composition of claim 16, wherein the drug transporter is ABCF1.

19. The composition of claim 16, wherein the composition promotes a 20% greater apoptosis rate in cancer cells compared to chemodrug alone.

20. The composition of claim 16, wherein the composition promotes a 40% greater apoptosis rate in cancer cells compared to chemodrug alone.
